(19) United States
(12) Patent Application Publication
(10) Pub. No.: US 2009/0118738 A1
(43) Pub. Date: May 7, 2009

(54) NEEDLESS APPLICATOR SYSTEM AND METHOD FOR APPLICATION OF MEDICAMENT TO THE BACK OF AN EYE

(76) Inventor: Scott J. Gerondale, Mission Viejo, CA (US)

Correspondence Address: ALLERGAN, INC., 2525 DUPONT DRIVE, T2-7H, IRVINE, CA, 92612-1599, US

(21) Appl. No.: 11/577,732

(22) PCT Filed: Oct. 26, 2005
(86) PCT No.: PCT/US05/38703

§ 371 Date: Apr. 23, 2007

Related U.S. Application Data
(60) Provisional application No. 60/623,533, filed on Oct. 29, 2004.

(51) Int. Cl. A61F 9/007

(52) U.S. Cl. 606/107

(57) ABSTRACT

The present invention includes a method of intraocular delivery by needless injection of a medicament through the sclera of an eye and into a back of the eye as well as instruments for such delivery.

### CROSS REFERENCE TO RELATED APPLICATIONS

This is a national stage application under 35 U.S.C. § 371 of PCT application PCT/US2005/038703, filed Oct. 26, 2005, which claims the benefit of U.S. Provisional Application Ser. No. 60/623,533 filed Oct. 29, 2004 which is incorporated by reference in its entirety herein.

The present invention generally relates to method of ophthalmic administration. More specifically, the present invention relates methods of intrascleral injection of therapeutic or diagnostic materials.

Delivering therapeutic or diagnostic agents to the posterior segment of the eye, especially to the retina, macula, etc., is difficult. Topical instillation of a medicament to the front of the eye such as by eye drops, generally does not provide sufficient amounts of the agent to the posterior portion of the eye. This is due in part to poor diffusion through the various layers as well as the natural clearing processes encountered.

Accordingly, introducing effective amounts of a medicament to, for example, the retina via topical instillation is generally not possible given the distance and tissue between the sclera and the retina. Another potential shortcoming with topical instillation is that the composition tends to be quickly removed from the eye by tears and other natural clearing processes. The resulting short duration of contact can further limit the likelihood of an appreciable amount of the agent reaching the posterior segment.

Systemic delivery of an agent to the posterior segment of the eye such as by oral administration, is limited by the blood-retinal barrier. The barrier limits the size and amount of agents that can reach the choroid and retina. Moreover, because the medicament is systemically delivered, the dosage is limited so as not to provide a toxic dose of the agent to other parts of the body.

Inserts which release the agent over time onto or into the eye may be utilized to deliver a medicament to the posterior segment of the eye. The medicament that is released from the insert can diffuse through the sclera and into the eye. However, little if any of the medicament reaches the posterior segment of the eye for much the same reasons as topical instillation.

Implants are devices similar to inserts but they are surgically placed within the eye. Accordingly, implants bring the risk of infection and other problems due to the invasive procedure required.

Intraocular delivery instruments in accordance with the prior invention provide for intrascleral system of medicament.

### SUMMARY OF THE INVENTION

The present invention includes a method of intraocular delivery by needless injection of a medicament through the sclera of an eye and into a back of the eye as well as instruments for such delivery.

### BRIEF DESCRIPTION OF THE DRAWINGS

The advantages and features of the present invention will be better understood by the following description when considered in conjunction of the accompanying drawings, in which:

FIG. 1 is a perspective view of the needleless injector in accordance with the present invention generally showing a housing and injection head and a vial along with a trigger;

FIG. 2 is a side view and partial cross section of the mechanism disposed within the housing shown in FIG. 1 which generally includes: an injection head, a vial, a bolt, a cocking stud, a valve tube a mainspring, a hammer, a sear, a valve seat, a trigger, a cup seal, a rear spring and a gas cartridge;

FIG. 3 is a cross sectional view of the injector shown in FIG. 2 with the bolt shown in a cocked position ready for injection of medicament through an injection head;

FIG. 4 is a view similar to that shown in FIG. 3 with the bolt shown in an uncocked position after dispensing of medicament through the injection head;

FIG. 5 is a front perspective view of another embodiment in accordance with the present invention generally showing a frame, barrel, vial, a cylinder for supporting a plurality of cartridge and a trigger mechanism;

FIG. 6 is a rear perspective view of the injection;

FIG. 7 is a cross-sectional view of the injector shown in FIG. 5 showing a port near the barrel distal end, a bolt disposed within a barrel bore along with a hammer and spring mechanism, a bolt being shown in a second position before firing of a cartridge and ejection of medicament through the injection head;

FIG. 8 is a cross-sectional view similar to that shown in FIG. 7 illustrating depression of the trigger and movement of a gas cartridge aligned with the hammer and bore for driving the bolt forward as with hereinafter described in greater detail;

FIG. 9 is a view taken along the line 9-9 of FIG. 7;

FIG. 10 is a perspective view of yet another embodiment of a needleless spring injector in accordance with the present invention generally showing a tubular casing having a bottom half shell and a top half shell;

FIG. 11 is a perspective view similar to that shown in FIG. 10 there showing the top half shell in an open position during cocking of a spring shown disposed within the tubular casing;

FIG. 12 is a cross sectional view of the needleless spring injector shown in FIGS. 10-11 illustrating a spring in a cocked position, a plunger ratchet, push rod, syringe and trigger;

FIG. 13 is a cross sectional view similar to that shown in FIG. 12 illustrating movement of the ratchet one step causing forward motion of the plunger by the spring and ejection of medicament to the injection head;

FIGS. 14-18 illustrate the cocking mechanism of the present invention effected through hinged opening of the top half shell of the injector.

FIG. 19 is a perspective view of a further embodiment of a needleless push-release injector in accordance with the present invention generally showing a housing, cocking ring;

FIG. 20 is a perspective view of the present invention illustrating internal components including a vial, check valve, piston, spring, along with the cocking ring and injection head as shown in FIG. 19;

FIG. 21 is a cross-sectional view of the injector shown in FIGS. 19-20, more particularly illustrating components of the present invention in which the spring is held in the compressed state by a sear prior to release for injection of medicament through an injection head into the patients skin, not shown in FIG. 21;

FIG. 22 is a cross-sectional view similar to that shown in FIG. 21 illustrating release of the piston upon pressure being applied to the injection head by the skin of a user;

FIG. 23 is a perspective view of the injection head cylinder piston and cocking ring illustrated;

FIG. 24 is a perspective view of still another embodiment in accordance with the present invention generally showing a housing, a vial for containment of a medicament, an injection head, a cocking grip and a trigger;

FIG. 25 is a cross sectional view of the injector shown in FIG. 24 more specifically showing a chamber disposed within the housing, a piston slidably disposed within the housing with a piston head fitted within the chamber, and having a stem with a spring disposed therearound along with a sear for holding the piston in a second position with the spring compressed;

FIG. 26 is a cross sectional view similar to FIG. 25 showing release of the piston with the spring forcing the piston head through the chamber in order to force medicament through the injection head;

FIG. 27 is a cross sectional view similar to FIGS. 25 and 26 showing cocking of the spring and withdrawal of medicament from the vial into the chamber for subsequent injection;

FIG. 28 is a perspective view of a twist-set embodiment injector in accordance with the present invention generally showing a housing, an injection head, a rotatable grip and a trigger;

FIG. 29 is a plan view of the injector shown in FIG. 28 partially broken away to illustrate a vial for containment of a medicament, a piston, a one-way valve, a mainspring, and a check valve;

FIG. 30 is a cross sectional view of the injector shown in FIGS. 28 and 29 more specifically showing a chamber disposed within the housing, a piston slidably disposed within the housing with a piston head fitted within the chamber, and having a stem with a spring disposed therearound along with a sear for holding the piston in a second position with the spring compressed;

FIG. 31 is a cross sectional view similar to FIG. 30 showing release of the piston with the spring forcing the piston head through the chamber in order to force medicament through the injection head;

FIG. 32 is a cross sectional view similar to FIGS. 30 and 31 showing cocking of the spring and withdrawal of medicament from the vial into the chamber for subsequent injection by rotation of the grip;

FIG. 33 is a perspective view of an inline needleless injector in accordance with the present invention generally showing a casing, an injection head disposed at a casing distal end, and a gas line;

FIG. 34 is a view of the embodiment of FIG. 33 in accordance with the present invention in partial cross section generally showing a hollow casing having a chamber disposed therein along with a piston, gas manifold, drug manifold, vial, duckbill valve, and cocking stud;

FIG. 35 is a cross section of the embodiment shown in FIGS. 33-34 of the generally showing a chamber piston biased by a spring;

FIG. 36 is a cross sectional view corresponding to the embodiment shown in FIG. 35 however utilizing application of gas pressure for driving a piston forward for forcing medicament through the injection head;

FIG. 37 is a perspective view of a needleless solenoid driven injector in accordance with the present invention generally showing a housing, an injection head, and an attached power cord;

FIG. 38 is a cross sectional view of the injector shown in FIG. 37 illustrating a cylinder, a piston disposed therein along with a solenoid driven plunger operated by a trigger, check valves for controlling flow of medicament through a manifold and dip tube for withdrawal of medicament from a vial disposed within the housing; and

FIGS. 39-40 are cross sectional views illustrating operation of the needleless solenoid driven injector in accordance with the present invention.

### DETAILED DESCRIPTION

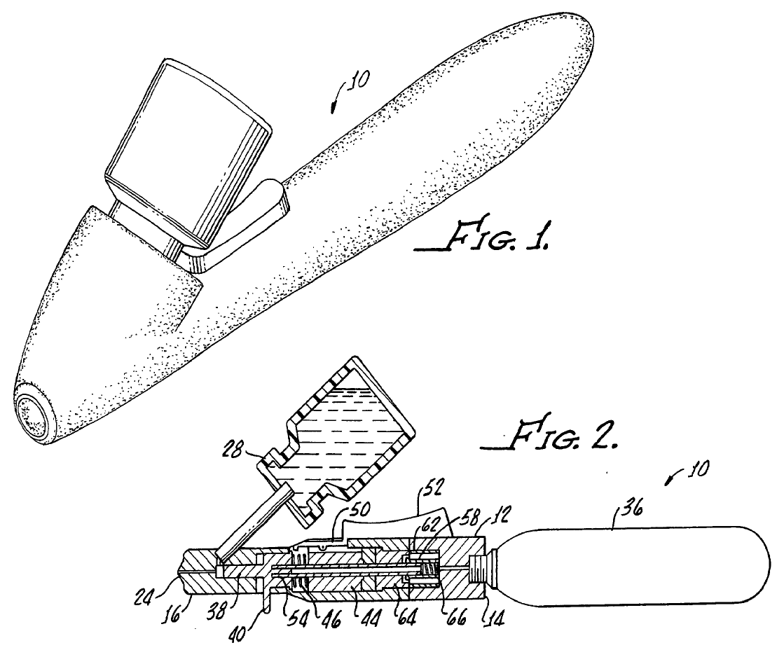

With reference to FIGS. 1-4, an intraocular delivery instrument 10 for practice of the method of the present invention generally includes a barrel 12 having a proximal end 14 and a distal end 16 and a bore 20 therethrough. An injection head 24 is disposed at the barrel distal end 16 which is in fluid communication with the bore 20 for the injection of a medicament therethrough.

A vial 28 is provided for containing a medicament, with the vial 28 being in fluid communication with the bore 20 through a port 32 near the barrel distal end 16. Preferably, the vial 28 is removably attached to the barrel 12.

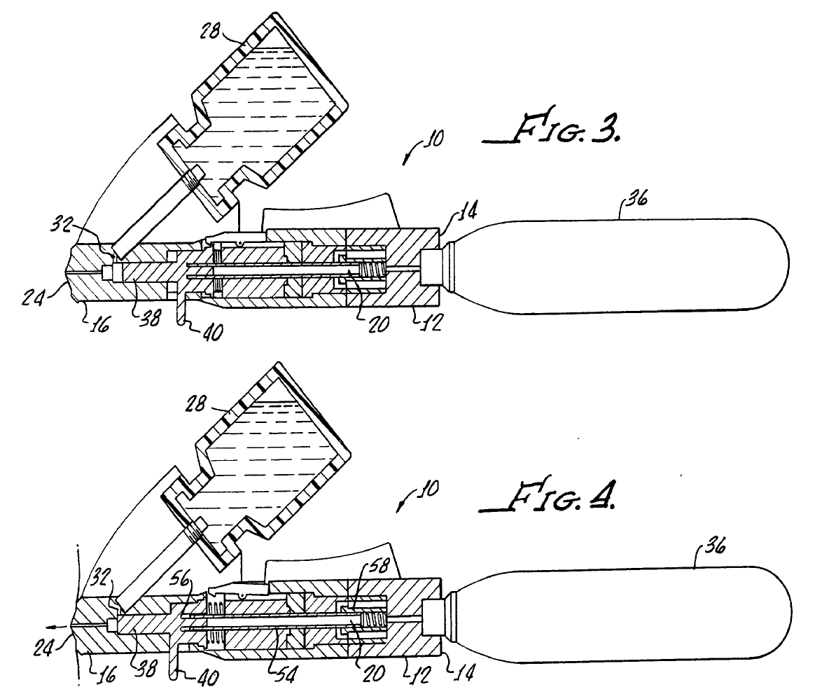

A gas cartridge 36 is disposed at the barrel proximal end 14. A bolt 38 is provided and disposed in the bore 20 for reciprocal movement therein from a first position closing the port 32 (FIG. 4) to a second position (FIG. 3) opening the port 32 for release of medicament into the bore 20. The bolt 38 is sized and fitted to provide sufficient seal between the bolt 38 and the bore 20 in order to draw medicament from the vial 28 into the bore 20 upon movement of the bolt 38 from the first position to the second position and to force medicament through the injection head 24 upon movement of the bolt 38 from the second to the first position as will be hereinafter described in greater detail.

A cocking stud 40 is provided for enabling manual movement of the bolt 38 from the first to the second position. In this cocking movement, medicament is drawn into the bore 20 between the bolt 38 and the injection head 24 through the port 32.

A hammer 44 is provided and disposed for reciprocal movement within the bore 20 between the bolt 38 and the barrel proximal end 12. A mainspring 46 is disposed between the bolt 38 and the hammer 44 for forcing the hammer 44 toward the barrel distal end 16.

A sear 50 is provided for releasably holding the bolt 38 in the second position with the mainspring 46 compressed between the bolt 38 and the hammer 44 and a trigger 52 is disposed in an operative relationship with the sear 50 for releasing the spring 46 in order to drive the hammer 44 toward the barrel distal end 16.

A valve tube 54 is provided and disposed within the bore 20 with a distal end 56 thereof attached to the bolt 38 and a proximal end 58 attached to the gas cartridge 36. The valve tube 54 extends through the hammer 44 and mainspring 46.

A cup seal 62 includes a valve seat 64 for controlling gas flow from the cartridge 36 into the valve tube 54. In that regard, a rear spring 66 is provided for releasably sealing the cup seal 62 against the valve seat 64, the cup seal 62 being open momentarily by impact of the hammer 44 after release by the sear 50 in order to allow gas to flow through the valve tube 54 to force the bolt 38 to the first position which ejects medicament through the injection head 24. The rear spring 66 thereafter closes the cup seal 62 against the valve seat 64 after the momentary opening.

In operation, when the bolt 38 is not cocked a forward portion 68 blocks entry of medicament into the bore 20 extending through the barrel 12. In order to cock the bolt 38, the cocking stud 40 is moved rearwardly pushing against the mainspring 46 in order that the bolt 38 butts up against the hammer 44. This motion opens the port 32 between the vial 28 and the bore 20 and draws medication into the bore 20 between the injection head 24 and the bolt 38. The sear 50 catches the bolt 38 as the bolt 38 is pushed against the hammer 44 and this binds the bolt 38 and the hammer 44 together so they move as one unit.

For injecting a medicament, the trigger 52 is pushed in order to release the sear 50 which releases the hammer 44 from the bolt 38 and the mainspring 48 propels the hammer 44 rearwardly in order to push momentarily onto the valve tube 54 which propels the valve tube 54 backward with sufficient force to overcome the rear spring 66. At that point, gas pressure is released from the gas cartridge 36 through the tube 54 which causes the bolt 38 to rapidly move forward and eject medicament disposed between a front of the bolt 38 and the injection head 24 through the injection head 24.

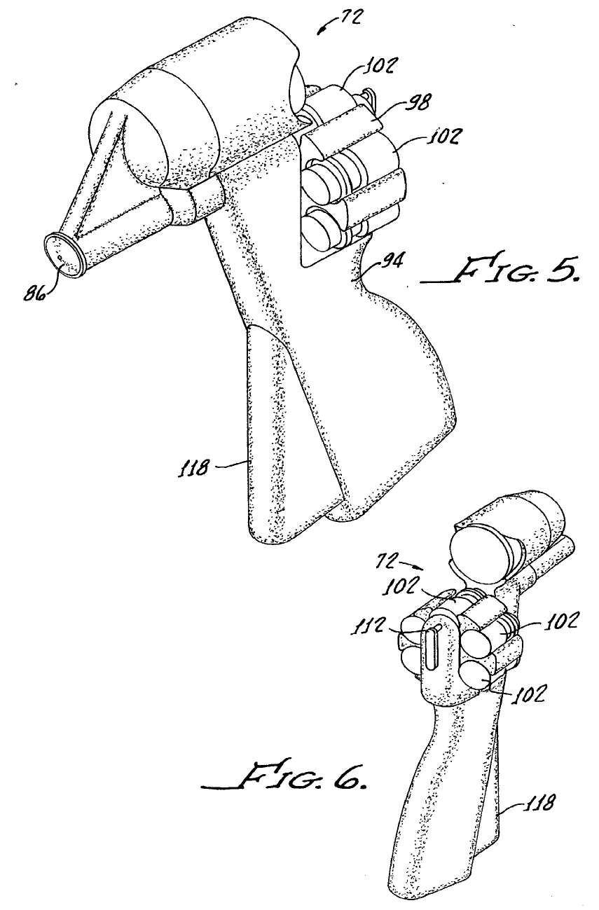

With reference to FIGS. 5-8 another embodiment of an intraocular delivery instrument 72 in accordance with the present invention generally includes a barrel 76 having a proximal end 78 and a distal end 80 and a bore 82 therethrough with an ejection head 86 disposed at the barrel distal end 80 in fluid communication with the bore 82.

A vial 90 is provided for containing a medicament with the vial 90 being in fluid communication with the bore 82 through a port 92 near the barrel distal end 80. Preferably, the vial 90 is removably attached to the barrel 76.

A frame 94 is provided for supporting the barrel 76 and a cylinder 98 mounted for rotation at the barrel proximal end 78 with the cylinder 98 having a plurality of chambers 100 therein for receiving gas cartridges 102.

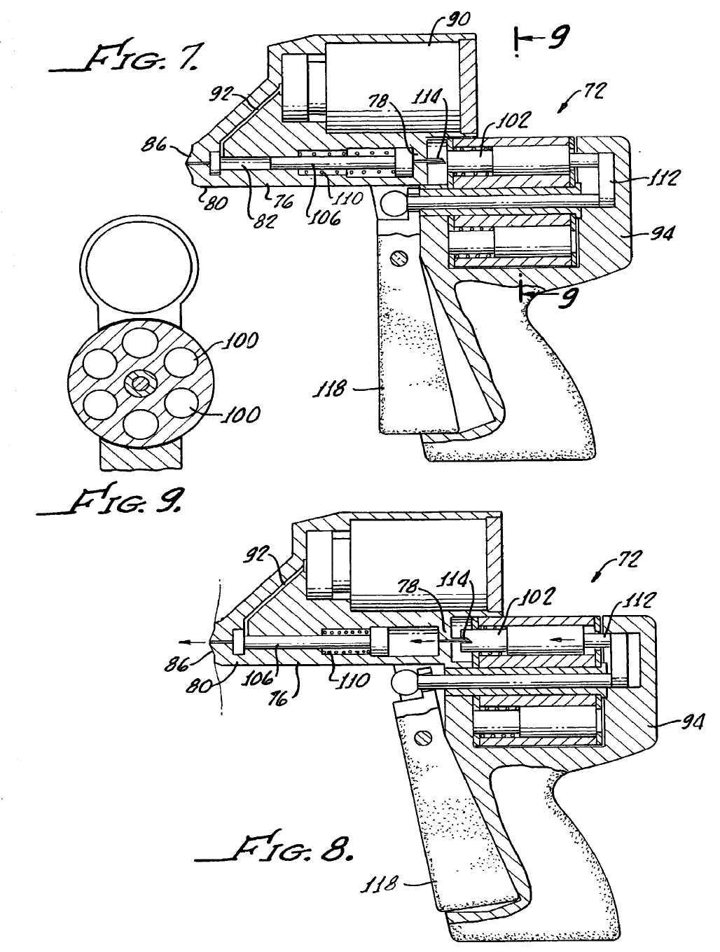

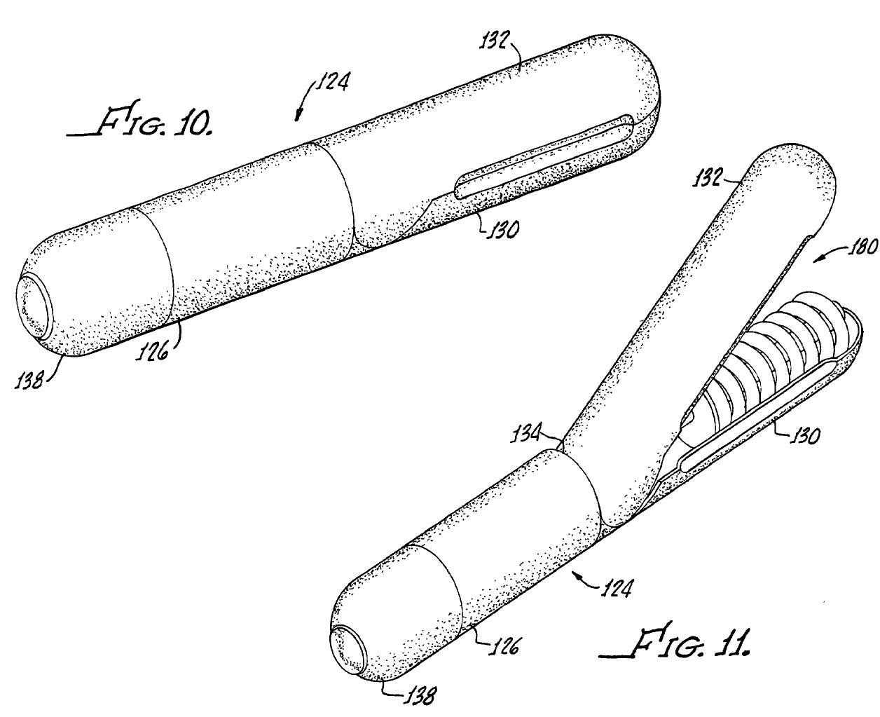

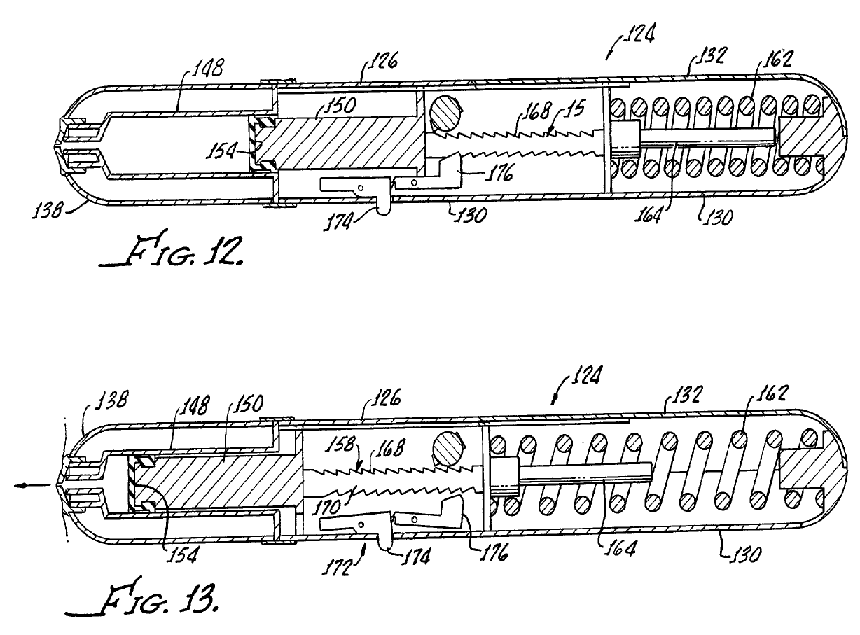

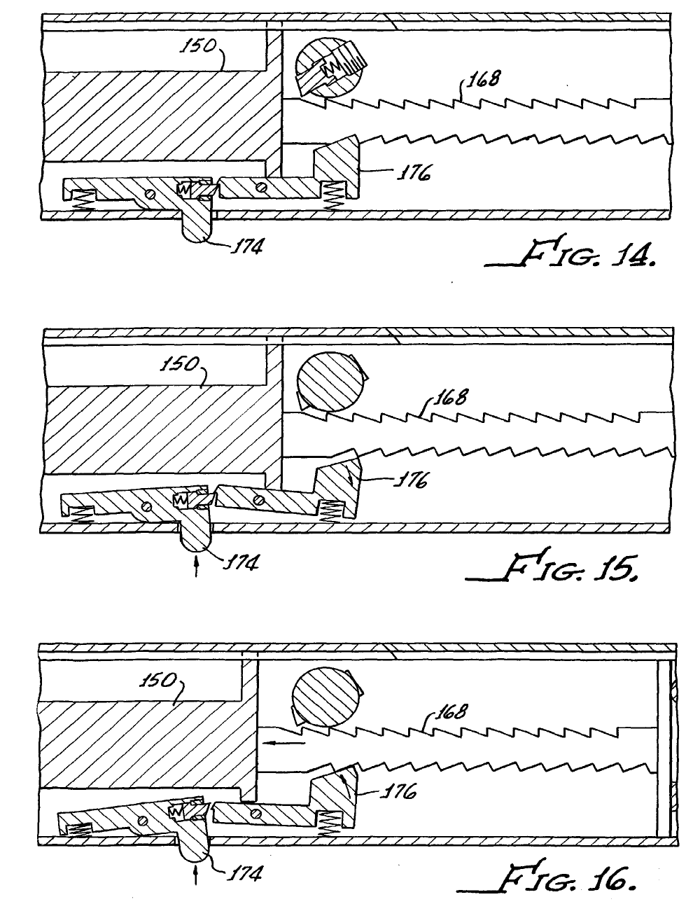

A bolt 106 is disposed in the bore 82 for reciprocating movement from a first position closing the port (FIG. 8) to a second position opening the port (FIG. 7) for release of medicament into the bore 82. The bolt 106 is fitted within the bore 82 in order to draw medicament from the vial 90 into the bore 82 upon movement of the bolt 106 from the first position to the second position and to force medicament through the injection head 86 upon movement of the bolt 106 from the second to the first position.

A spring 110 is disposed adjacent to the bolt 106 for driving the bolt 106 from the first to the second position and a hammer 112 is disposed between the bolt 106 and the cylinder 98 which includes a firing pin 114 for puncturing a gas cartridge 102 aligned with the hammer 112 and bore 82 by rotation of the cylinder 98.

A trigger 118 is pivotally attached to the frame 94 for forcing the aligned gas cartridge 102 into the hammer 112 for puncture of the aligned gas cartridge 102 in order to drive the bolt 106 from the second position to the first position and thereby force medicament in the bore 82 through the injector head 86. After release of pressure the spring 110 forces the bolt 106 from the first position to the second position thus drawing additional medicament into the bore 82 from the vial 90 for repeated injection.

It should be appreciated that the cylinder 98 may be manually rotated to align the bore 82 and hammer 112 or a pawl and ratchet arrangement (not shown) may be provided in order to rotate the cylinder 98 prior to a movement of a gas cartridge 102 into engagement of the hammer 112 for release of pressure therein.

Yet another embodiment 124 in accordance with the present invention generally includes a tubular casing 126 having a bottom half shell 130 extending from a rear end 132 thereof. A top half shell 132 is hingeably 134 attached to the casing rear end 132 for covering the bottom half shell 130.

A nose cap 138 is removably attached to a front end 142 of the tubular casing 126 and a injection head 144 is disposed in an end of the nose cap 138.

A syringe 148 is releasably disposed within the tubular casing 126 and is in fluid communication with the injection head 144. Slidably disposed within the tubular casing 126 is a plunger 150 which engages one end 154 of the syringe 148 for injecting medicament therefrom through the injection head 144. A push rod 158 is provided between the bottom shell 130 and top half shell 132 for slidably moving the plunger 150. A spring 162, disposed about a rear portion 164 of the push rod 158 is provided for driving the push rod 158.

A ratchet 168 is disposed on a forward portion 170 of the push rod 158 and a manually operated trigger 172, pivotally mounted within the tubular casing 126, and including a button 174 extending outwardly from the tubular casing 126 is provided in order to enable manual depression thereof and a sear 176 is also provided for releasably engaging the ratchet 168.

A cocking mechanism 180 engages the push rod 158 and the top half shell 132 compresses the spring 162 upon hinge 134 opening of the top half shell 132 from the bottom half shell 130.

The spring 162 is retained in a compressed state by engagement of the sear 176 with the ratchet 168 and the depression of the button 174 releases the sear 176 from the ratchet 168 enabling expansion of the spring 162 to drive the push-rod 158 and the plunger 150 in a forward direction in a metered manner to effect the ejection of the medicament from the syringe 148 through the injection head 144.

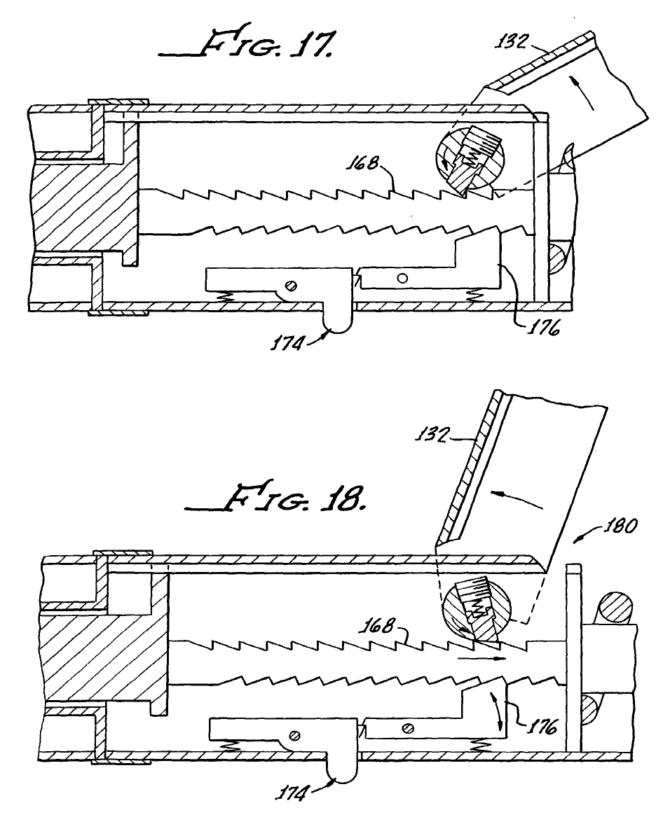

FIGS. 14-16 illustrate the depression of the button 174 initiating a single step forward movement of the ratchet 168 and push rod 158 for single dose ejection of a medicament.

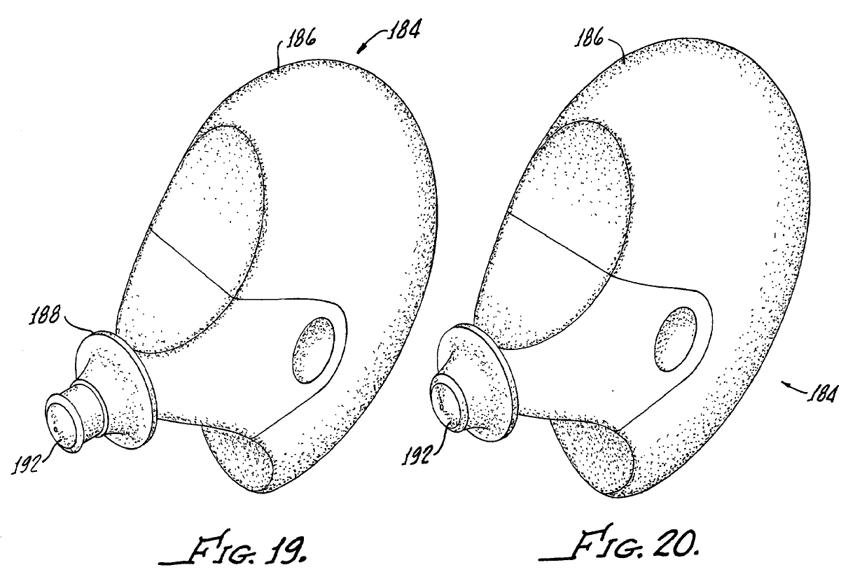

FIGS. 17 and 18 show a cocking mechanism 180 for engaging the push rod ratchet 168 and compressing the spring 162 on hinged opening of the top half shell 132 from the bottom half shell 130. The spring 162 is retained in a compressed state by engagement of the sear 176 with the ratchet 168.

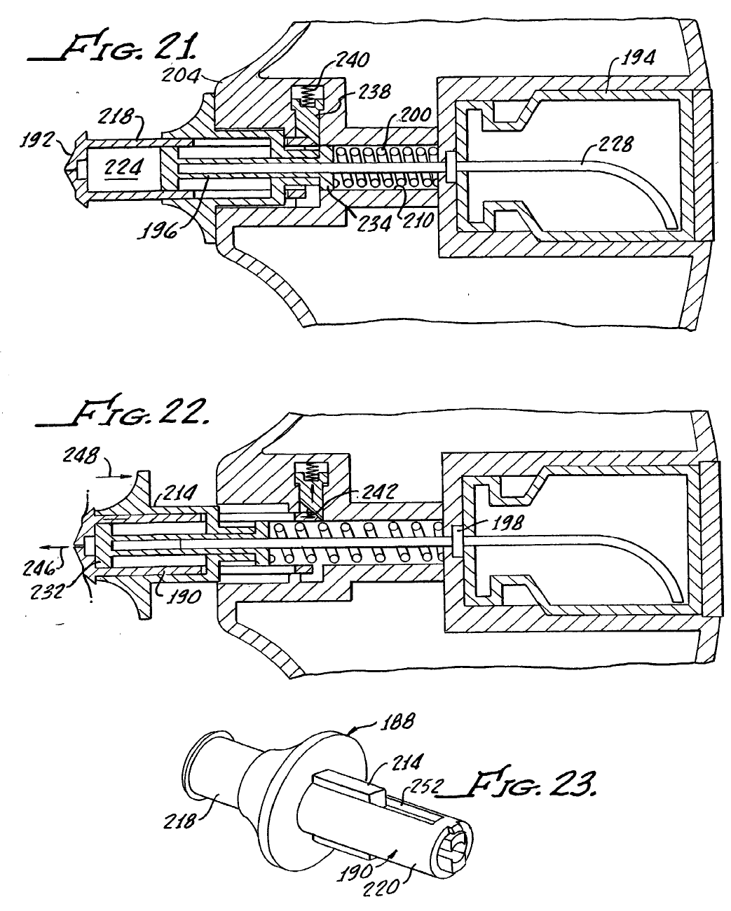

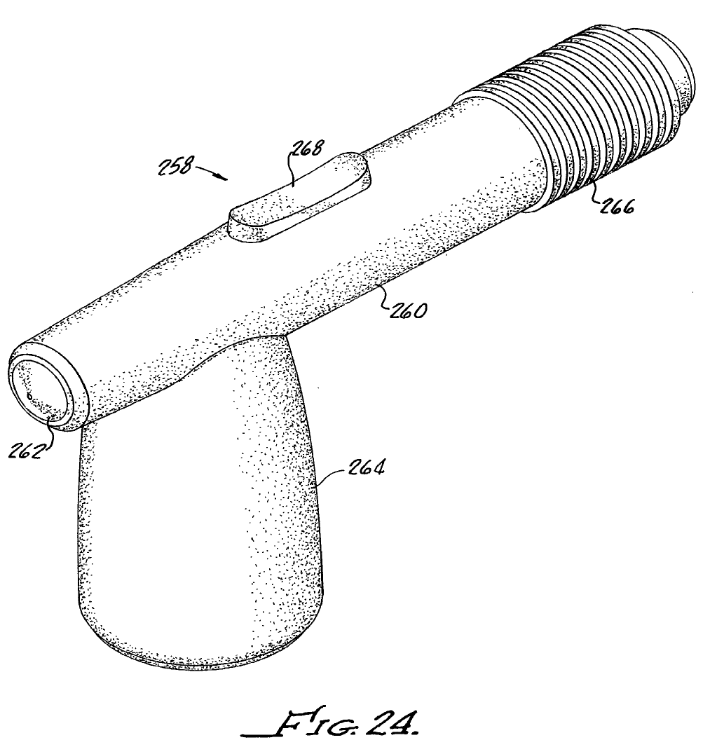

With reference to FIGS. 19-23 there is shown a needleless push-release injector 184 in accordance with the present invention generally showing a housing 186 along with a cocking ring 188, a cylinder 190, injection head 192, a vial 194, piston 196, valve 198, and a spring 200. With reference to FIGS. 21 and 22, the housing 186 includes a distal end 204 and a proximal end 206 with a bore 210 therein. The cocking ring 188 extends from the housing distal end 204 and includes a rear portion 214 disposed within the housing bore 210.

The cylinder 190 is slidably disposed within the cocking ring 188 and includes a front end 218 and a rear end 220 with a chamber 224 disposed proximate the front end 218. The vial 194 for containing a medicament is replaceably disposed in the housing 186 and is in fluid communication with the chamber 224 through a dip tube 228 and check valve 198.

The injection head 192 is disposed at the cylinder front end 218 and in fluid communication with the chamber 224. The injection head 192 may be of any suitable design.

The piston 196 is slidably disposed within the cocking ring 188 and includes a front head 232 slidably disposed within the cylinder 224 and a rear head 234 slidably disposed within the housing bore 210.

The front head 232 is fitted to the cylinder 190 in order to draw medicament from the vial 194 into the chamber 224 through the one-way valve 198 upon movement of the piston 196 from a first position shown in FIG. 22 to a second position shown in FIG. 21. Movement of the piston 196 from the first position to the second position forces medicament from the chamber 224 through the injection head 192.

The main spring 200 is disposed between the housing proximal end 206 the piston rear head 234 and a sear 218 is provided for releasably holding of the piston 196 in the second position with the main spring 200 compressed. The sear 238 includes a sear spring 240 and a tapered end 242 which provides releasable engagement with the piston rear head 234.

In use, the injector head 192 is pressed against skin (not shown) which causes rearward motion of the cylinder 190 and the cylinder rear end causes compression of the sear spring 240 which releases the piston rear head 234 and piston 196, thus causing the spring 200 to force the piston front head 232 into the chamber 224 forcing medicament through the injection head 192, as indicated by the arrow 246.

To re-cock the device the cocking ring 188 is moved in the direction of the arrow 248, thus moving the piston 196 and re-engagement of the sear 238 with the piston rear head 234.

As illustrated in FIG. 23, the cocking ring may include guides 252 for preventing rotation thereof.

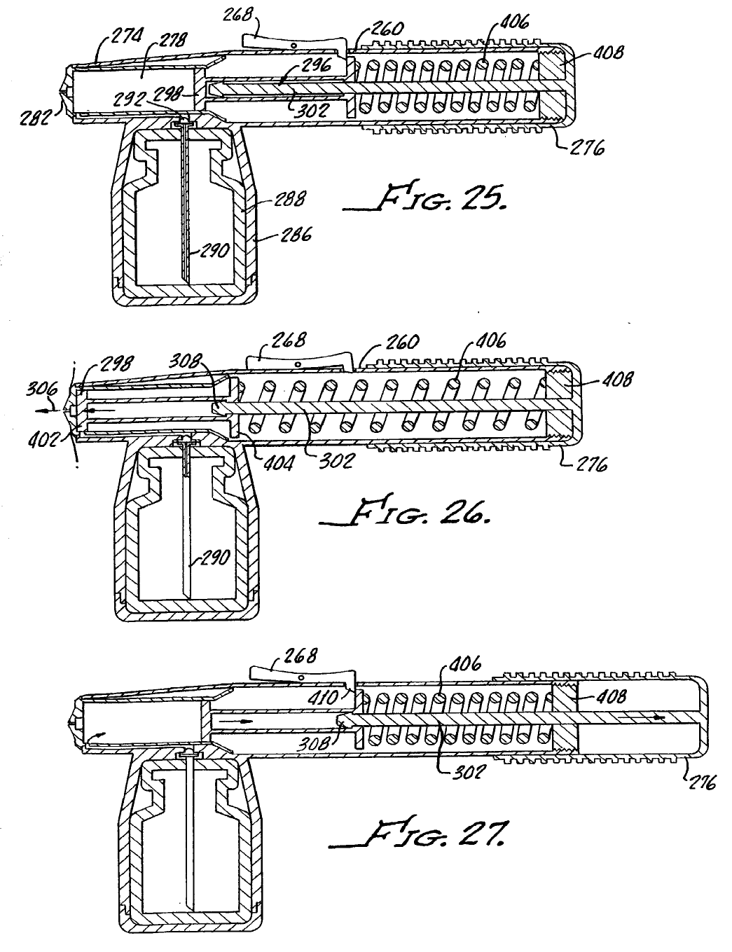

With reference to FIG. 24, there is shown still another embodiment 258 generally including a housing 260, an injection head 262, a vial 264 for containing a medicament, a cocking grip 266, and a trigger 268.

As shown in FIGS. 25-27, the housing 260 includes a distal end 274 and a proximal end 276 with a chamber 278 disposed within the housing at the distal end 274.

The injection head 282, which may be of conventional design, is disposed at the housing distal end 274 and is in fluid communication with the chamber 278.

A vial 286 may support a replaceable container 288 containing a medicament, not shown, as hereinabove referenced and is in fluid communication with the chamber 278 through a dip tube 290 and a one-way valve 292.

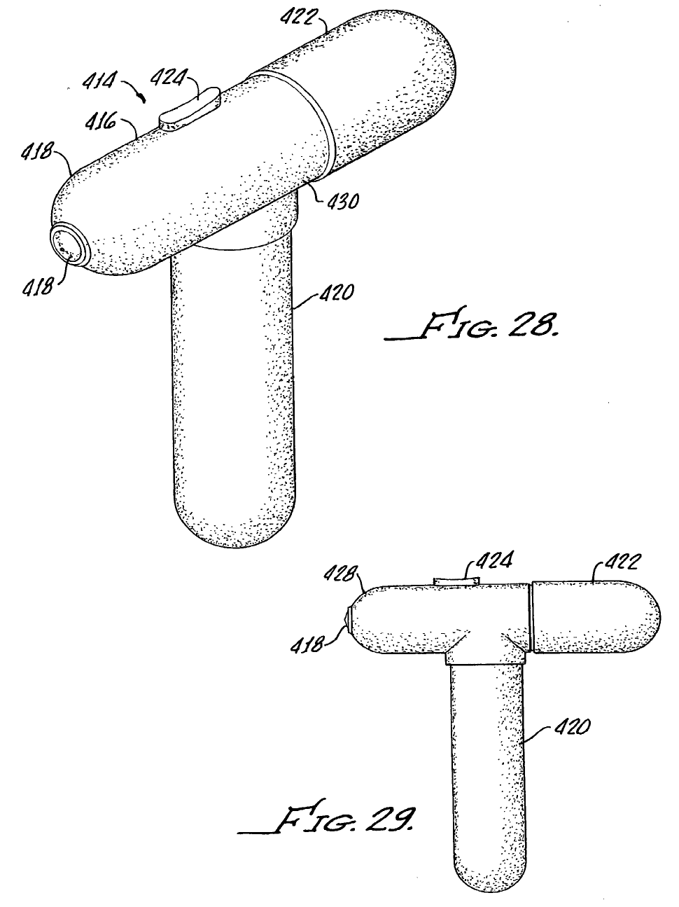

A piston 296 is slidably disposed within the housing 260 and includes a piston head 298 slidably disposed within the chamber 278 and a separately moveable stem 302 disposed in the housing 260. The piston head 298 is fitted within the chamber 278 in order to draw medicament from the vial 286 into the chamber 278 through the one-way valve 292 and dip tube 290 upon movement of the piston head 298 from a first position, as shown in FIG. 26, to a second position shown in FIG. 27.

Medicament is forced through the injection head 282 by movement of the piston head from the second position, as shown in FIG. 25, to the first position as shown in FIG. 26 with the injected medicament being indicated by the arrow 306. A head 308 on the piston stem 302 couples the stem 302 in a slidable manner with the piston head 298 between a fore plate 402 an aft plate 404 of the piston head 298.

As shown in FIGS. 25-27, a spring 406 disposed around the piston stem 302 between the aft plate 404 and an end plate 408 which is compressed by the cocking grip 266 which is slidably disposed over the housing proximal end 276. Reciprocal movement of the cocking grip 266 causes compression of the spring 406, as illustrated in the figures. A sear 410 is provided for releasably holding the piston 296 in the second position with the spring 406 compressed by engagement with the aft plate 404, as shown in FIGS. 25 and 26.

The trigger 268 is pivotally disposed on a housing 260 and in an operational relationship with the sear 410 for releasing the spring 406 in order to drive the piston 296 along with fore plate 402 to the first position, thus ejecting a metered dose of medicament determined by the chamber 278 volume.

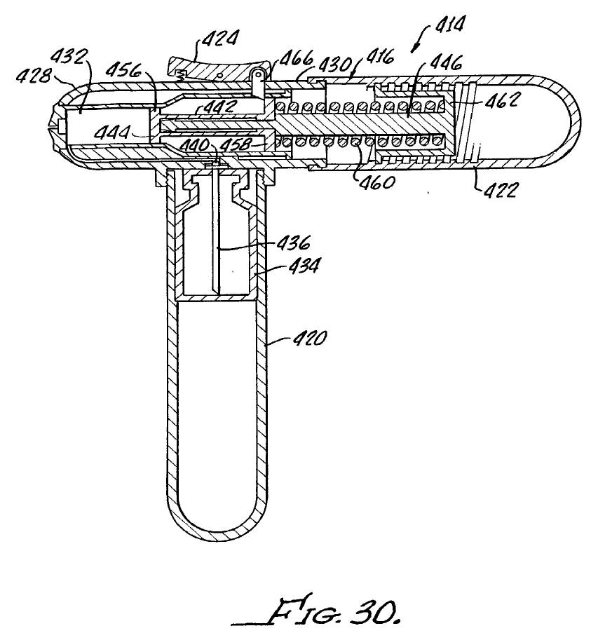

With reference to FIGS. 28 and 29, there is shown a twist-set needleless injector generally including a housing 416, an injection head 418, a vial 420 for containing a medicament, a rotatable grip 422 and a trigger 424.

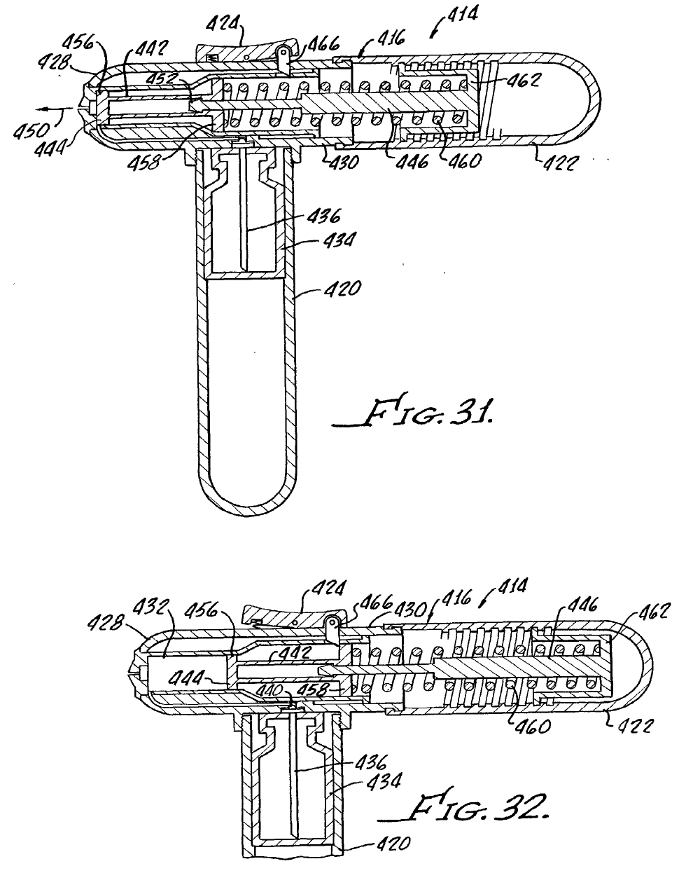

As shown in FIGS. 30-32, the housing 416 includes a distal end 428 and a proximal end 430 with a chamber 432 disposed within the housing at the distal end 428.

The injection head 418, which may be of conventional design, is disposed at the housing distal end 428 and is in fluid communication with the chamber 432.

The vial 420 may support a replaceable container 434 containing a medicament, not shown, as hereinabove referenced and is in fluid communication with the chamber 432 through a dip tube 436 and a one-way valve 440, for example a duckbill valve.

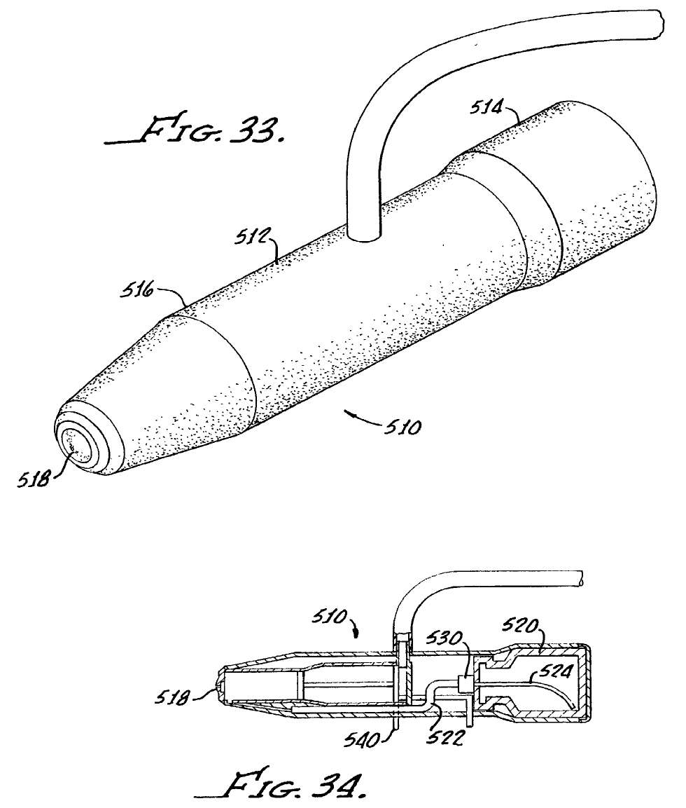

A piston 442 is slidably disposed within the housing 416 and includes a piston head 444 slidably disposed within the chamber and a moveable stem 446 disposed in the housing 416. The piston head 444 is fitted within the chamber 432 in order to draw medicament from the vial 420 into the chamber 432 through the one-way valve 440 and dip tube 436 upon movement of the piston head 444 from a first position, as shown in FIG. 31, to a second position shown in FIG. 32.

Medicament is forced through the injection head 418 by movement of the piston head 444 from the second position, as shown in FIG. 31, to the first position as shown in FIGS. 30 and 32 with the injected medicament being indicated by the arrow 450. A head 452 on the piston stem 446 couples the stem 446 in a slidable manner with the piston head 444 between a fore plate 456 an aft plate 458.

As shown in FIGS. 30-31, a spring 460 disposed around the piston stem 446 between the aft plate 458 and an end plate 462 which is compressed by the rotating grip 422 which is threadably disposed at the housing proximal end 430. Rotational movement of the cocking grip 422 causes compression of the spring 460, as illustrated in the figures. A sear 466 is provided for releasably holding the piston 442 in the second position with the spring 460 compressed by engagement with the aft plate 458, as shown in FIG. 30.

The trigger 424 is pivotally disposed on the housing 416 and in an operational relationship with the sear 466 for releasing the spring 460 in order to drive the piston 442 along with fore plate 456 to the first position, thus ejecting a metered dose of medicament determined by the chamber 432 volume.

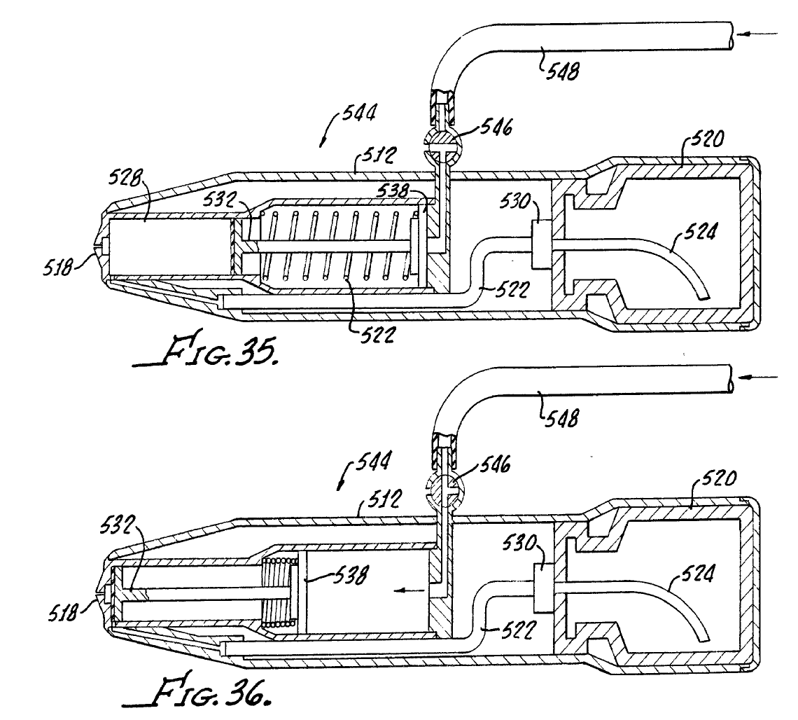

With reference to FIGS. 33 and 34, there is shown an inline needleless injector 510 having a hollow casing 512 with a proximal end 514 and a distal end 516, an injection head 518 is disposed at the casing distal end 516 and a vial 520 for containing a medicament, is disposed within the casing 512 at the proximal end 514 thereof.

A drug manifold 522 or line communicates with the vial 520 through a dip tube 524 and a chamber 528 for transport of medicament from the vial 520 into the chamber 528. A one-way valve 530, such as, for example, a duckbill valve, disposed in the drug manifold 522 prevents transport of medicament into the vial 520 from the chamber 528.

A piston 532 is provided and slidably disposed within the chamber 528 for forcing medicament through the injection head 518 and withdrawing medicament from the vial 520 into the chamber 528 through the manifold 522. A gas manifold 536 is provided behind a proximal end 538 of the piston 532 for introducing gas from a line behind the piston proximal end 538 in order to drive the piston 532 toward the casing distal end 516 thereby forcing medicament through the injection head 518.

In the embodiment 510, a cocking stud 540 may be provided in order to move the piston 532 toward the casing proximal end 514 in order to withdraw medicament from the vial 520 and into the chamber 528 for subsequent injections.

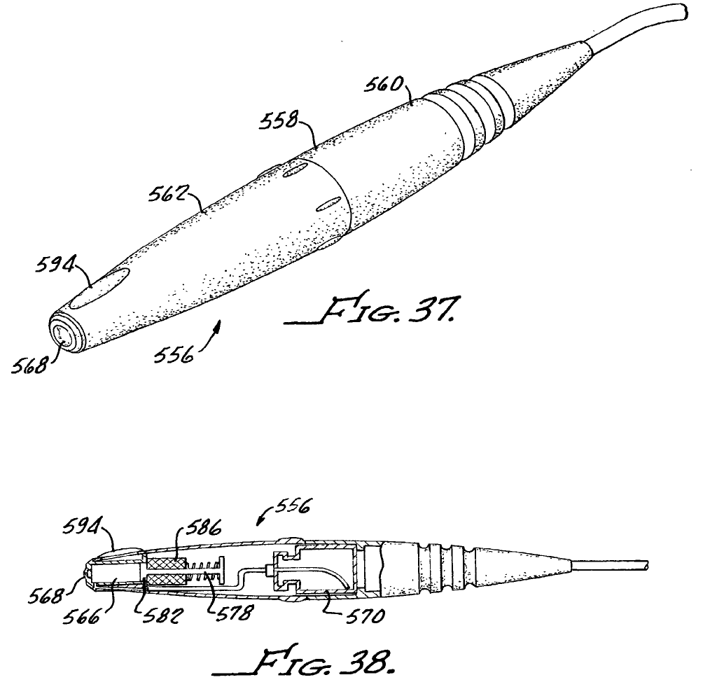

With reference to FIGS. 35 and 36, there is shown another embodiment 544 of an inline needleless injector in accordance with the present invention. Common reference characters represent identical or substantially similar elements, as hereinabove described in connection with the injector as shown in FIGS. 33 and 34.

A manually operated gas valve 546, not shown, in FIGS. 33 and 34, is disposed proximate the casing 512 for enabling control of a gas through a gas line 548 into the manifold 522. In the embodiment, a spring 552 is provided as a means for moving the piston 532 toward the casing proximal end 514 after discharge of medicament through the injection head 518 in order to withdraw medicament from the vial 520 through the drug manifold 522.

Operation of the injector is illustrated in FIGS. 35 and 36 respectively. In FIG. 35, the chamber 528 which is filled with medicament, is ready for injection with the piston disposed with its proximal end 538 adjacent the gas manifold 536. Opening of the gas valve 546 causes gas to drive the piston toward the casing distal end 516 forcing medicament through the injection lead 518, as shown in FIG. 36.

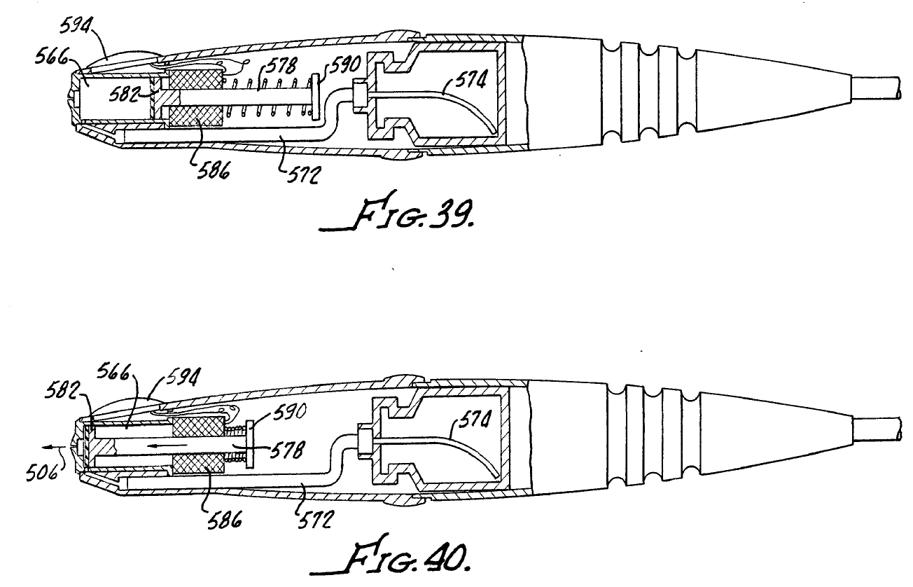

With referenced to FIGS. 37 and 38, there is shown a needleless injector 556 in accordance with the present invention including a housing 558 having a proximal end 560 and a distal end 562 with a cylinder 566 disposed in the housing 558 at the distal end 562 thereof along with an injection head 568 disposed at one end of the cylinder 566. A vial 510 for containing a medicament, is disposed within the housing 558 at the proximal end 560 thereof. A manifold 522 interconnects the cylinder with the vial for transport of medicament from the vial 510 to the cylinder 566 utilizing a dip tube 524 disposed within the vial 510.

A plunger 578 includes a first end, or piston, 582 slidably disposed within the cylinder 566 for forcing medicament in the cylinder 566 through the injection head 568 upon movement of the plunger 578 toward the injection head 568 and for withdrawing medicament from the vial 510 into the cylinder 566 upon movement of the plunger 578 away from the injection head 568. As hereinafter described, the plunger 578 is operable, or moveable, through the application of a magnetic flied provided by a solenoid 586. The solenoid 586 is disposed around the plunger 518 between the first end 582 and a second end 590 of the plunger 518. The solenoid 586 is disposed in an operational manner for forcing the plunger 578 toward the injection head 568 upon application of electrical current controlled by a trigger switch 594 interconnected with a power supply such as, for example, a 110-volt AC line.

A spring 598 is disposed around the plunger 578 between the solenoid 586 and the second end 590 for forcing a plunger 578 away from the injection head 568 after electrical current is not applied to the solenoid 586 in order to withdraw medicament from the vial 510 into the cylinder 566, as hereinabove noted.

As more clearly shown in FIGS. 39 and 40, the vial 510 may be removable from the housing 558 through a coupling 602 between the proximal end 560 and distal end 562 of the housing 558.

Operation of the device is illustrated in FIGS. 39 and 40, with FIG. 39 illustrating the injector in a charged state with the plunger 518 disposed at a proximal end 560 of the cylinder 566.

Upon energizing the solenoid 586 through the application of electrical current controlled by the trigger switch 594, the plunger 578 is forced to a distal end of the cylinder 566 as shown in FIG. 40 forcing medicament through the injector head 568 as indicated by the arrow 606. Electrical wiring, (not shown), conventionally interconnects trigger 594 and solenoid 586.

After injection of medicament through the injection head 568, power is removed from the solenoid 586 through the switch 594 enabling the spring 598 to force the plunger 578 away from the injector head 568 with the first end, or piston, 582 thereof withdrawing to the proximal end of the cylinder 566.

Although there has been hereinabove described specific intraocular delivery instruments and a method in accordance with the present invention for the purpose of illustrating the manner in which the invention may be used to advantage, it should be appreciated that the invention is not limited thereto. That is, the present invention may suitably comprise, consist of, or consist essentially of the recited elements. Further, the invention illustratively disclosed herein suitably may be practiced in the absence of any element which is not specifically disclosed herein. Accordingly, any and all modifications, variations or equivalent arrangements which may occur to those skilled in the art, should be considered to be within the scope of the present invention as defined in the appended claims.

What is claimed is:

1. (canceled)

2. An intraocular delivery instrument comprising:
a barrel having a proximal and a distal end and a bore therethrough;
an injection head disposed at the barrel distal end in fluid communication with said bore;
a vial for containing a medicament, said vial being in fluid communication with said bore through a port near the barrel distal end;
a gas cartridge disposed at the barrel proximal end; and
a bolt disposed in said bore for reciprocating movement therein, from a first position closing said port to a second position opening said port for release of medicament into said bore, said bolt being fitted to said bore in order to draw medicament from said vial into said bore upon movement of said bolt from the first position to the second position, and to force medicament through the injection head upon movement of the bolt from the second to the first position.

3-12. (canceled)

13. The instrument according to claim 1 wherein said vial removably attached to said barrel.

14. The instrument according to claim 1 further comprising a cocking stud for enabling manual movement of said bolt to the second position.

15. The instrument according to claim 1 further comprising a hammer disposed for reciprocal movement in said bore between said bolt and the barrel proximal distal end.

16. The instrument according to claim 15 further comprising a mainspring disposed between said bolt and said hammer for forcing said hammer toward the barrel distal end.

17. The instrument according to claim 16 further comprises a sear for releasably holding said bolt in the second position with mainspring compressed between said bolt and said hammer.

18. The instrument according to claim 17 further comprising a trigger disposed in an operational relationship with said sear for releasing the spring in order to drive the hammer toward the barrel distal end.

19. The instrument according to claim 15 further comprising a valve tube disposed within said bore and having a distal end attached to said bolt and a proximal end, attached to the gas cartridge, said valve tube extending through said hammer.

20. The instrument according to claim 19 further comprising a cup seal including a valve seat for controlling gas flow from the cartridge into said valve tube.

21. The instrument according to claim 20 further comprising a rear spring for releasably sealing the cup seal against said valve seat, said cup seal being opened momentarily by impact of said hammer to allow gas to flow through said valve tube to force said bolt to the first position in order to eject medicament through said injection head, said rear spring closing the cup seal against the valve seal after said momentary opening.

22. An intraocular delivery instrument comprising:
a barrel having a proximal and a distal end and a bore therethrough;
an injection head disposed at the barrel distal end in fluid communication with said bore;
a vial for containing a medicament, said vial being in fluid communication with said bore through a port near the barrel distal end and removably attached thereto;
a gas cartridge disposed at the barrel proximal end; and
a bolt disposed in said bore for reciprocating movement therein, from a first position closing said port to a second position opening said port for release of medicament into said bore, said bolt being fitted to said bore in order to draw medicament from said vial into said bore upon movement of said bolt from the first position to the second position, and to force medicament through the injection head upon movement of the bolt from the second to the first position;

23. The instrument according to claim 22 further comprising a cocking stud for enabling manual movement of said bolt to the second position.

24. The instrument according to claim 22 further comprising a hammer disposed for reciprocal movement in said bore between said bolt and the barrel proximal distal end.

25. The instrument according to claim 24 further comprising a mainspring disposed between said bolt and said hammer for forcing said hammer toward the barrel distal end.

26. The instrument according to claim 25 further comprises a sear for releasably holding said bolt in the second position with mainspring compressed between said bolt and said hammer.

27. The instrument according to claim 26 further comprising a trigger disposed in an operational relationship with said sear for releasing the spring in order to drive the hammer toward the barrel distal end.

28. The instrument according to claim 24 further comprising a valve tube disposed within said bore and having a distal end attached to said bolt and a proximal end, attached to the gas cartridge, said valve tube extending through said hammer.

29. The instrument according to claim 28 further comprising a cup seal including a valve seat for controlling gas flow from the cartridge into said valve tube.

30. The instrument according to claim 29 further comprising a rear spring for releasably sealing the cup seal against said valve seat, said cup seal being opened momentarily by impact of said hammer to allow gas to flow through said valve tube to force said bolt to the first position in order to eject medicament through said injection head, said rear spring closing the cup seal against the valve seal after said momentary opening.
